(19) United States
(12) Patent Application Publication
(10) Pub. No.: US 2023/0127034 A1
(43) Pub. Date: Apr. 27, 2023

(54) MOUNTABLE VIRTUAL REALITY STATION AND SYSTEM

(71) Applicant: David BLACK, Annapolis, MD (US)

(72) Inventor: David BLACK, Annapolis, MD (US)

(21) Appl. No.: 17/857,981

(22) Filed: Jul. 5, 2022

Related U.S. Application Data
(60) Provisional application No. 63/218,021, filed on Jul. 2, 2021.

(51) Int. Cl. G06F 3/01; G06F 3/0488

(52) U.S. Cl. CPC G06F 3/011; G06F 3/0488

(57) ABSTRACT

A mountable virtual reality station and system include a unit body having a plurality of walls including a back wall and a front wall. The back wall is configured for connection with a structure, such as a wall of a room or booth. The unit further includes a touch screen and headset mounting display arranged on the front wall, and a power source connected with the unit body. Preferably, the unit includes a system indicator display, wherein the system indicators are at least one of a power indicator, a Wi-Fi indicator, and a Bluetooth indicator. The unit also preferably includes a virtual reality headset tether connected with the unit body configured for connection with a virtual reality headset.

This application claims the benefit of U.S. Provisional Patent Application No. 63/218,021 filed Jul. 2, 2021, the entire contents of which is incorporated herein by reference.

### BACKGROUND OF THE DISCLOSURE

The present disclosure relates generally to virtual reality (VR) devices, and specifically to a mountable station for housing virtual reality equipment.

Virtual reality equipment typically involves a headset including video screens and speakers, among other devices. Virtual reality units can further include motion sensing devices to add to the experience and more accurately portray real-world situations.

When not in use, virtual reality equipment is often stored in a portable container, such as a bag or pouch. This is beneficial for an individual who owns the equipment, but it is not ideal for multi-user equipment, such as virtual reality equipment used by third parties in public settings.

Thus, there is a need for a more permanent form of storage for public-use devices, as well as a need to provide a storage device that displays the equipment to potential public users. Moreover, there is a need for a storage container that sanitizes publicly-used virtual reality equipment and which provides connections with third-party devices, such as phones, tablets, and biometric devices, to name a few.

### SUMMARY OF THE DISCLOSURE

Accordingly, it is an object of the present disclosure to provide a mountable virtual reality station and system including a unit body having a plurality of walls including a back wall and a front wall. The back wall is configured for connection with a structure such as a wall of a room or booth. The unit further includes a touch screen and headset mounting display arranged on the front wall, and a power source connected with the unit body. Preferably, the unit includes a system indicator display, wherein the system indicators are at least one of a power indicator, a Wi-Fi indicator, and a Bluetooth indicator.

In one embodiment, the unit further includes a virtual reality headset tether connected with the unit body and configured for connection with a virtual reality headset.

In a second embodiment, the unit further includes a pair of side walls with an access panel and/or a cooling device arranged on one of the side walls.

In another embodiment, the unit further includes a mounting assembly configured for connection with a railing system.

In yet another embodiment, the unit further includes an eye tracking device and motion sensors configured to track movement.

In another embodiment, the unit further includes digital memory, at least one computer processor, and machine readable instructions configured to integrate the station and system with devices, for instance personal biometric devices.

In a further embodiment, the unit further includes a sterilization unit arranged on the front wall including a plurality of walls defining an inner chamber. The sterilization unit preferably includes a UVC light configured to clean and store a virtual reality headset and a locking mechanism to secure a headset.

### BRIEF DESCRIPTION OF THE FIGURES

Other objects and advantages of the disclosure will become apparent from a study of the following specification when viewed in the light of the accompanying drawing, in which:

FIG. 1 is a front perspective view of a mountable virtual reality unit station according to the disclosure;

FIG. 2 is a side view of the embodiment of FIG. 1; and

FIG. 3 is a perspective view of the embodiment of FIG. 1.

### DETAILED DESCRIPTION

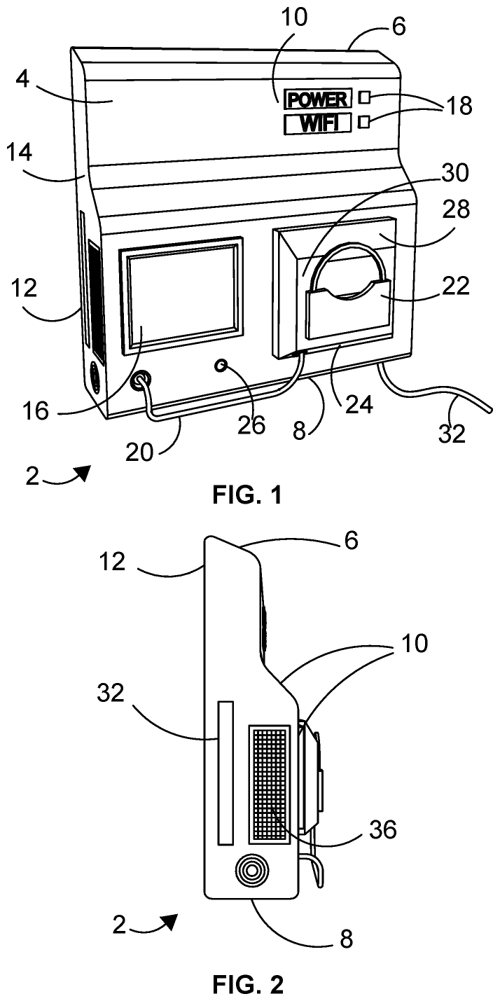

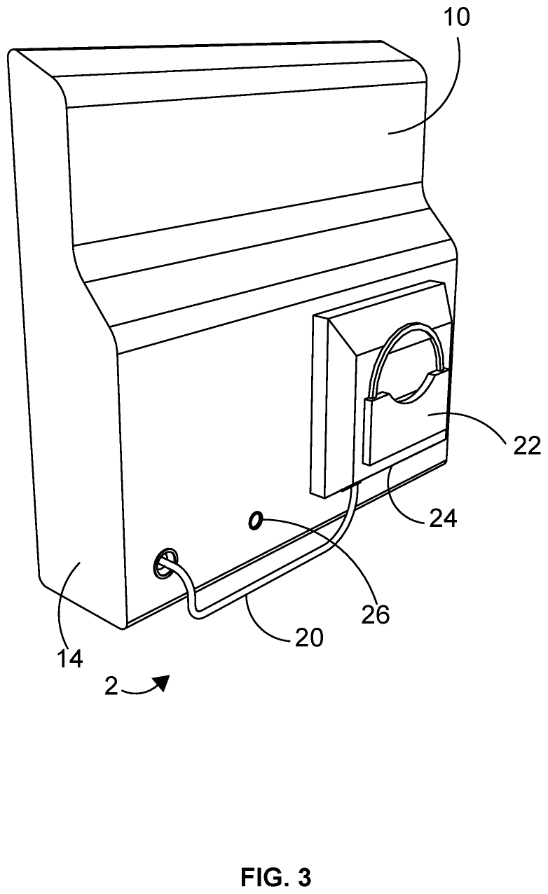

FIGS. 1-3 show a preferred embodiment of the mountable virtual reality station and system 2 of the present disclosure, which includes a hard casing unit 4 that houses hardware elements and includes top 6, bottom 8, front 10, rear 12 and side 14 walls. The housing unit is adapted to be mountable with a wall or booth, preferably via a mounting assembly (not shown), by attaching the rear wall with a railing system that can be connected to a wall or booth.

The front wall 10 includes a touchscreen 16 with device navigation for users to select software applications, enter personal information, or otherwise customize their VR experience. The front wall further includes displays 18 that indicate power, Wi-Fi, and other connected device statuses (e.g. Bluetooth) to provide users with visibility into the security connection and operating status of the system.

A tether 20 connects a VR headset 22 to the front wall 10 of the main housing unit 2 to provide both a security measure and power source for the VR head mounted display (HMD) unit 24. The HMD unit includes at least one display, lenses and semi-transparent mirrors embedded in eyeglasses, and a visor or helmet. Preferably, the unit also includes eye tracking technology and motion sensors for tracking a user's movement, for instance hand movement.

The station and system further includes hardware, such as a CPU (not shown), arranged within the housing unit 2, which includes software programming to run the system and integrate the station equipment with wearable devices, preferably via a Bluetooth connection. There is also a camera and/or motion sensor 26 that provides hand tracking integration for user interface navigation and selection capabilities while wearing the VR headset 22.

A sterilization and housing unit 28 for the VR headset 22, which contains a chamber 30 to store and sanitize the VR headset between uses, is arranged on the front wall 10 of the housing unit 2. The chamber is preferably accessible via a lockable door (not shown). The headset is stored in this chamber and sanitized by a UVC light (not shown) arranged within the chamber of the sterilization and housing unit. Other methods for sanitizing the headset are contemplated, for instance by use of a sanitizing mist and/or sanitizing wipes.

The station and system are preferably powered via a 110v external power cord connection 32. However, wireless methods of power are also contemplated, such as a rechargeable battery.

To access the hardware, such as the CPU, touch screen, motion sensor, etc., for repairs and/or replacement, an access panel 34 including a removable cover is located on one of the side walls 14. The station and system also preferably includes a cooling device 36 such as a venting panel or fan to reduce the temperature of the unit.

To use the station and system, the mounting assembly is connected with a wall or booth, preferably via a mounting rail, and the power cord 32 is connected with a power outlet. A user can then interact with the touchscreen 16 to choose a specific VR program, remove the VR headset 22 from the housing unit 28, wear the headset and use the VR software. While using the system, the user will have the option of connecting user devices with the system via Bluetooth, Wi-Fi or another wireless/wired connection. This will allow the user to synchronize user data and/or programs with the system as desired by the user or required by specific programs. When the user is finished, the VR headset 22 is once again placed in the housing unit 28 and sanitized for future use. The system can be powered down or remain on for another user. If desired, the station and system can be removed from the wall or booth it is mounted on and moved to another location for mounting and use.

Although the above description is in reference to a particular embodiment, it is to be understood that the embodiment is merely illustrative of the principles and applications of the present disclosure. It is therefore to be understood that numerous modifications may be made to the illustrative embodiment and that other arrangements may be devised and employed without departing from the spirit and scope of the present disclosure.

What is claimed is:

1. A mountable virtual reality station and system comprising:
(a) a unit body having a plurality of walls including a back wall and a front wall, said back wall being configured for connection with a structure;
(b) a touch screen arranged on said front wall;
(c) a headset mounting display arranged on said front wall; and
(d) a power source connected with said unit body.

2. The mountable virtual reality station and system defined in claim 1, and further comprising a virtual reality headset tether connected with said unit body configured for connection with a virtual reality headset.

3. The mountable virtual reality station and system defined in claim 1, and further comprising a pair of side walls, one said side wall including at least one of an access panel and a cooling device.

4. The mountable virtual reality station and system defined in claim 1, and further comprising a mounting assembly configured for connection with a railing system.

5. The mountable virtual reality station and system defined in claim 1, and further comprising a display arranged on said front wall, said display including system indicators, wherein the system indicators are at least one of a power indicator, a Wi-Fi indicator, and a Bluetooth indicator.

6. The mountable virtual reality station and system defined in claim 1, and further comprising an eye tracking device and motion sensors configured to track movement.

7. The mountable virtual reality station and system defined in claim 1, and further comprising digital memory, at least one computer processor, and machine readable instructions configured to integrate the station and system with devices.

8. The mountable virtual reality station and system defined in claim 1, and further comprising a sterilization unit arranged on said front wall including a plurality of walls defining an inner chamber, said sterilization unit being configured to clean and store a virtual reality headset.

9. The mountable virtual reality station and system defined in claim 8, said sterilization unit having a locking mechanism.

10. The mountable virtual reality station and system defined in claim 8, said sterilization unit including a UVC light.

11. The mountable virtual reality station and system defined in claim 1, said power source including one of a power cord and battery.
